(19) United States
(12) Patent Application Publication
(10) Pub. No.: US 2022/0148728 A1
(43) Pub. Date: May 12, 2022

(54) SYSTEM AND METHOD FOR ANALYZING STRESS OF USER AND MANAGING INDIVIDUAL MENTAL HEALTH, USING HMD DEVICE HAVING BIOSIGNAL SENSORS MOUNTED THEREIN

(71) Applicant: LOOXID LABS INC., Daejeon (KR)

(72) Inventors: Honggu LEE, Seoul (KR); Yongwook CHAE, Seoul (KR)

(21) Appl. No.: 17/433,610

(22) PCT Filed: Oct. 24, 2019
(86) PCT No.: PCT/KR2019/014073

§ 371 Date: Aug. 25, 2021

(30) Foreign Application Priority Data
Feb. 25, 2019 (KR) 10-2019-0021819

(51) Int. Cl. G16H 50/20; G16H 20/70

(52) U.S. Cl. CPC G16H 50/20; G16H 20/70

(57) ABSTRACT

The present invention relates to a method for analyzing stress and managing individual mental health, using a HMD device. The method includes a calibration step for generating standard stress information by calibrating biosignals received from a plurality of biosignal sensors; a stress measurement content performance step for generating a stress guiding screen, measuring biometric data of user via generated stress guiding screen, and calculating stress measurement information of user by comparing the measured biometric data with at least one among the standard stress information and biosignals; and a stress analysis content performance step for extracting characteristics from the biometric data, and predicting a stress index of the user on the basis of the extracted characteristics, in which the standard stress information includes an initial stress index, and reference value for specific emotions. Thus, by measuring biosignals using the biosignal sensors, the reliability of data enabling stress analysis may be increased.

### TECHNICAL FIELD

The present invention relates to a system and a method for analyzing stress of a user and managing individual mental health using an HMD device having biometric sensors mounted therein, and more particularly, to a system and a method for managing individual mental health using an HMD device with significantly improved reliability of stress level measurement using a plurality of biosignal sensors such as electroencephalogram (EEG), electrocardiogram (ECG), and eye sensors.

### BACKGROUND ART

Modern people receive physical or psychological stimulation every moment. When such stimulation is applied, the people will receive stress when emotions such as fears, anxiety or tensions for the stimulation are caused. In general, the stress may be defined in a variety of types, such as cognitive stress, business stress, and relationship stress.

In recent years, attempts to use biosignals are increasing to measure such stress objectively. These attempts are mainly analyzing HRV through an electrocardiogram (ECG) to evaluate the physiological responses to the stress to determine an action degree of an autonomic nervous system of the body.

In particular, as methods frequently used in the related art, there are most cases where the analysis was performed using a sensor (single modality). However, in the case of biosignals, an error is significantly large because signals having a significantly large level difference are detected according to peripheral situations or body internal situations of a user. Therefore, there is a problem that it is very difficult to perform accurate stress analysis based on the measured biosignals.

However, in relation to operating a plurality of sensors in combination, studies were considerably lacking on whether some sensors can derive accurate stress measurement in a complementary relationship.

Therefore, deep research is required on any combination of sensors for accurate stress derivation, and furthermore, it has been urgently required a system capable of detecting accurate stress using a characteristic that signals obtained from sensors with different types or properties contain various characteristics related to emotion.

### SUMMARY OF THE DISCLOSURE

An object of the present invention is to provide a system and a method for analyzing stress and managing individual mental health using a HMD device with improved reliability of data capable of analyzing stress by measuring biosignals using a plurality of biosignal sensors.

Another object of the present invention is to provide a system and a method for analyzing stress and managing individual mental health using a HMD device capable of effectively managing health by continuously measuring and feed-backing biosignals.

Yet another object of the present invention is to provide a system and a method for analyzing stress and managing individual mental health using a HMD device with increased user convenience by greatly improving accuracy of the stress level measurement using machine learning.

The objects of the present invention are not limited to the aforementioned objects, and other objects, which are not mentioned above, will be apparent to those skilled in the art from the following description.

One aspect of the present invention provides a method for analyzing stress and managing individual mental health, the method including: a calibration step for generating standard stress information by calibrating biosignals received from a plurality of biosignal sensors; a stress measurement content performance step for generating a stress guiding screen, measuring biometric data of a user via the generated stress guiding screen, and calculating stress measurement information of the user by comparing the measured biometric data with at least one among the standard stress information and the biosignals; and a stress analysis content performance step for extracting characteristics from the biometric data, and predicting a stress index of the user on the basis of the extracted characteristics, in which the standard stress information includes an initial stress index, and a reference value for a specific emotion. Thus, by measuring biosignals using the biosignal sensors, the reliability of data enabling stress analysis may be increased.

According to another feature of the present invention, in the stress analysis content performance step, the extracted characteristic may be substituted with a stress level to measure a stress index of the user, and the stress level may be calculated by the following Equation 1.

Stress level=EG1*Weeg1 . . . + . . . EEGN*WeegN+EGG1*Wecg1 . . . + . . . ECGM*WecgM+EYE1*Weye1 . . . + . . . EYEK*WeyeK [Equation 1]

(here, W represents a weight value of each of an EEG sensor, an ECG sensor, and an eye sensor.)

According to yet another feature of the present invention, in the stress analysis content performance step, at least one of the stress index and the emotion of the user may be analyzed by comparing a difference in the stress measurement information based on the standard stress information.

According to yet another feature of the present invention, in the stress analysis content performance step, the stress index may be predicted based on the extracted characteristics using a recurrent neural network (RNN) or a long short term memory (LSTM).

According to yet another feature of the present invention, the method may further include a stress relaxation content performance step of generating a stress relaxation content according to the stress analysis result, in which the stress relaxation content may be output in at least one form of sounds, images, and videos, and may be provided with a different content depending on a user or user's stress index.

According to yet another feature of the present invention, the plurality of biosignal sensors may include first and second biosignal sensors, and the method may further include receiving a first synchronization sensing signal induced from an event trigger signal and received from the first biosignal sensor; receiving a second synchronization sensing signal induced from the event trigger signal and received from the second biosignal sensor; and calculating time difference information of the first synchronization sensing signal and the second synchronization sensing signal based on a time when the event trigger signal is expressed, and synchronizing the first and second biosignal sensors based on the time difference information.

According to yet another feature of the present invention, the event trigger signal may be displayed on a display of the HMD device by randomly arranging familiar pictures and unfamiliar pictures.

According to yet another feature of the present invention, the event trigger signal may include a beep sound.

According to yet another feature of the present invention, the event trigger signal may be a blinking screen displayed on the display of the HMD device.

Another aspect of the present invention provides a system for analyzing stress and managing individual mental health, the system including: a HMD device which measures biosignals from a plurality of biosignal sensors; and a mental care server which receives the measured biosignals and calculates stress measurement information based on the received biosignals, in which the mental care server generates standard stress information by calibrating the biosignals, generates a stress guiding screen, measures biometric data of a user via the stress guiding screen, calculates stress measurement information of the user by comparing the measured biometric data with at least one of the standard stress information and the biosignals, extracts characteristics from the biometric data, and predicts a stress index of the user based on the extracted characteristics, in which the standard stress information includes an initial stress index, and a reference value for a specific emotion. Therefore, it is possible to efficiently manage health by continuously measuring and feed-backing the biosignals.

Details of other exemplary embodiments will be included in the detailed description of the invention and the accompanying drawings.

According to the present invention, it is possible to significantly improve reliability of stress analysis data by using a plurality of biosignal sensors including an ECG sensor, an EEG sensor, and an eye sensor. Therefore, due to low reliability, it was difficult to measure a stress index for a device in actual stress consultation work or medical stress analysis practice, but due to the improvement of the reliability according to the present invention, it is possible to measure a stress index using the device in the actual stress consultation work or the medical stress analysis practice.

Further, according to the present invention, it is possible to perform efficient mental health management through continuous monitoring of biosignals.

Further, according to the present invention, it is possible to greatly improve the accuracy of stress index measurement using machine learning.

Further, according to the present invention, it is possible to calibrate a time error between components in a system or a time error between different systems by performing time synchronization for a series of signals using at least two or more synchronization sensors.

The effects of the present invention are not limited by the foregoing, and other various effects are anticipated herein.

### BRIEF DESCRIPTION OF THE DRAWINGS

FIG. 1 is an overall schematic diagram of a system for analyzing stress and managing individual mental health using a HMD device according to an embodiment of the present invention.

FIG. 2 is a block diagram for describing a HMD device according to an embodiment of the present invention.

FIG. 3 is a block diagram for describing a mental care server according to an embodiment of the present invention.

FIG. 4 is an exemplary diagram for describing an electrocardiogram (ECG) according to an embodiment of the present invention.

FIG. 5 is an overall flowchart for describing a method for analyzing stress and managing individual mental health using a HMD device according to an embodiment of the present invention.

FIG. 6 is a flowchart for describing a method for performing an analysis content of a mental care server according to an embodiment of the present invention.

FIG. 7 is a diagram for describing a plurality of biosignal sensors attached to a HMD device according to an embodiment of the present invention.

FIGS. 8A to 8C are exemplary diagrams for describing a stress guiding screen according to an embodiment of the present invention.

FIG. 9 is a block diagram for describing a HMD device according to another embodiment of the present invention.

### DETAILED DESCRIPTION OF THE DISCLOSURE

The following description illustrates only a principle of the present invention. Therefore, although not clearly described or illustrated in the present specification, those skilled in the art can implement the principle of the present invention and invent various apparatuses included in the spirit and scope of the present invention. In addition, it is to be understood that all conditional terms and embodiments mentioned in the present specification are obviously intended only to understand a concept of the present invention in principle, and the present invention is not limited to embodiments and states particularly mentioned as such.

In the following description, ordinal expressions, such as first, second, etc., are intended to explain equivalent and independent objects, and it should be understood that there is no meaning of main/sub or master/slave in the order.

The above-mentioned objects, features, and advantages will become more obvious from the following detailed description associated with the accompanying drawings. Therefore, those skilled in the art to which the present invention pertains can easily practice the technical idea of the present invention.

The features of various exemplary embodiments of the present invention can be partially or entirely coupled or combined with each other and can be interlocked and operated in technically various ways to be sufficiently appreciated by those skilled in the art, and the exemplary embodiments can be implemented independently of or in association with each other.

Hereinafter, various exemplary embodiments of the present invention will be described in detail with reference to the accompanying drawings.

Configuration of HMD Device of System for Analyzing Stress and Managing Individual Mental Health

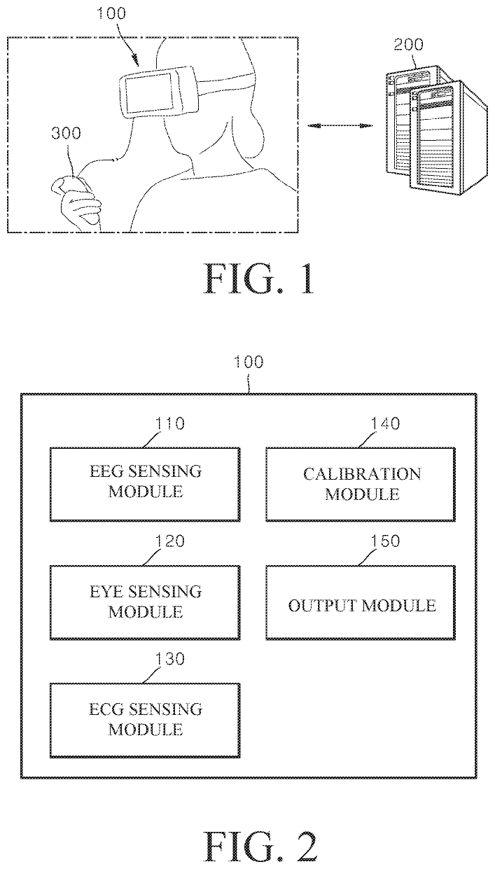

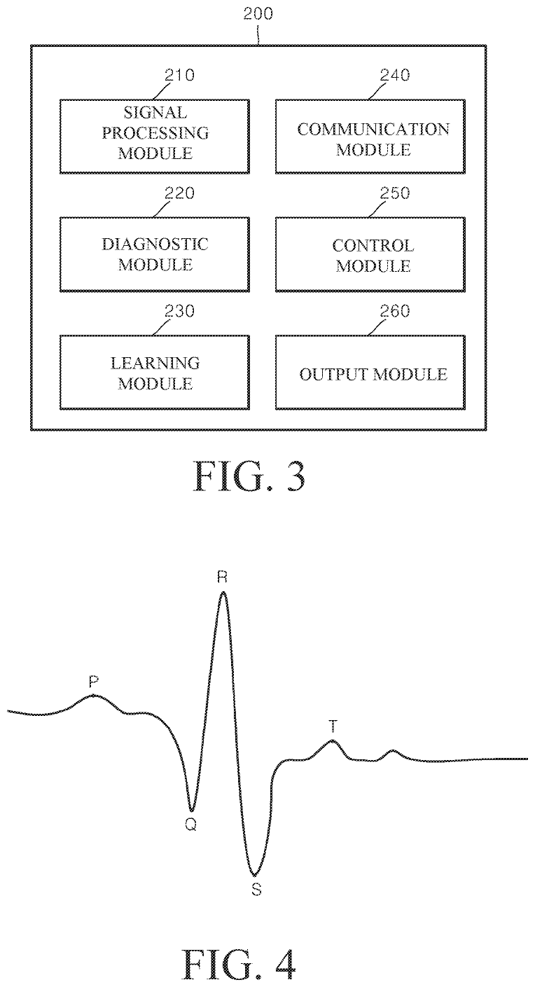

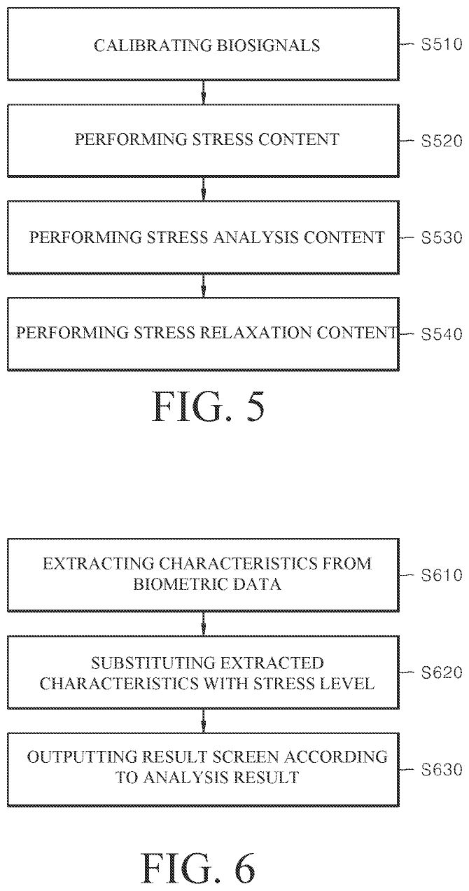

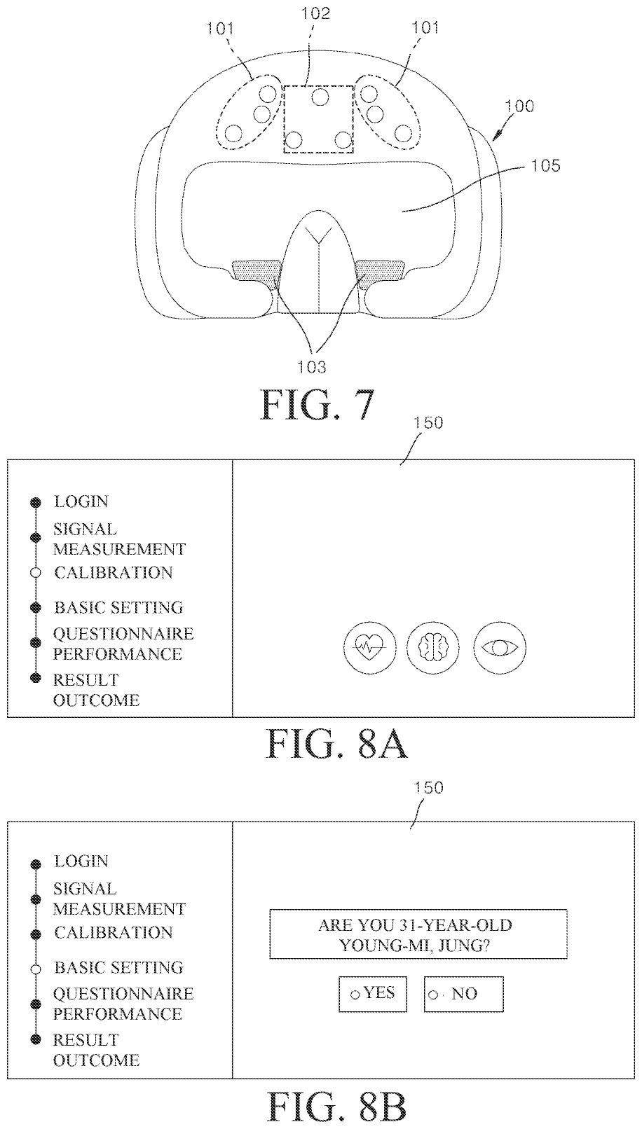

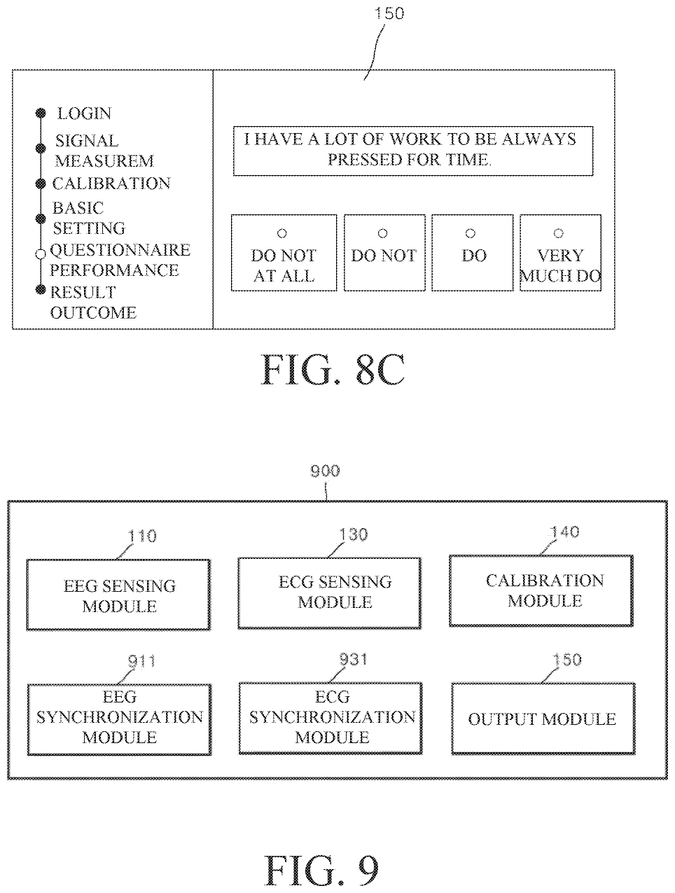

FIG. 1 is an overall schematic diagram of a system for analyzing stress and managing individual mental health using a HMD device according to an embodiment of the present invention. FIG. 2 is a block diagram for describing a HMD device according to an embodiment of the present invention. FIG. 3 is a block diagram for describing a mental care server according to an embodiment of the present invention. FIG. 4 is an exemplary graph for describing an electrocardiogram (ECG) according to an embodiment of the present invention. FIG. 5 is an overall flowchart for describing a method for analyzing stress and managing individual mental health according to an embodiment of the present invention. FIG. 6 is a flowchart for describing a method for performing an analysis content of a mental care server according to an embodiment of the present invention. FIG. 7 is a diagram for describing a plurality of biosignal sensors attached to a HMD device according to an embodiment of the present invention. FIGS. 8A to 8C are exemplary diagrams for describing a stress guiding screen according to an embodiment of the present invention.

Referring to FIG. 1, a system for analyzing stress and managing individual mental health includes an HMD device 100, biosignal sensors, and a mental care server 200.

The HMD device 100 includes biosignal sensors as various types of wearable devices that may be worn by a user and may sense biosignals of the user. Here, the biosignals may mean various signals that are generated from the user's body such as EEGs, eyes, motion of pupils, heart rate, and blood pressure of the user.

In the present invention, the HMD device 100 may be mounted on the head as a head-mounted display (HMD) to directly/indirectly present images to the user.

For example, the HMD device 100 may be a device in the form of supporting virtual reality including a display unit by itself such as Oculus® VR (virtual reality) and may also be a device in a similar form to Gear® VR used by mounting the display unit on the HMD mount. Alternatively, the HMD device 100 may be a device that supports augmented reality (AR) in the form of Google Glass® or Microsoft HoloLens®. Alternatively, the HMD device 100 may be a device that supports mixed reality such as Windows mixed reality (MR) or Odyssey Plus MR.

As illustrated in FIG. 2, the HMD device 100 may include an EEG sensing module 110 measuring an electroencephalogram from an electroencephalogram (EEG) sensor, an eye sensing module 120 measuring the motion of pupils from an eye sensor, an ECG sensing module 130 measuring an electrocardiogram from an electrocardiogram (ECG) sensor, a calibration module 140, and an output module 150. Meanwhile, in the present invention, as long as the EEG sensor, the eye sensor, and the ECG sensor may be easily in contact with a body site so as to measure biosignals of the user, the sensors are not limited only to the HMD device 100, but may be any type of wearable devices. For example, the HMD device 100 may be a headset, a smart watch, an earphone, a mobile device, etc.

As illustrated in FIG. 7, the biosignal sensors are attached to the HMD device 100 and include an ECG sensor 101, an EEG sensor 102, and an eye sensor 103.

The EEG sensing module 110 may sense the electroencephalogram of the user wearing the HMD device 100. The EEG sensing module 110 may include at least one electroencephalogram (EEG) sensor. When the user wears the HMD device, the EEG sensor attached to the HMD device 100 is in contact with a body site, such as a head or forehead where the EEG of the user can be measured, and then the EEG sensing module 110 may measure the EEG of the user. The EEG sensing module 110 may measure various frequencies of EEGs generated from the contacted body site of the user or electrical/optical frequencies changed according to an activation state of the brain.

However, since the EEGs are biosignals, there may be a difference according to a peripheral situation or an inner body situation of the user for each user or even in the same user. Thus, even in the same cognitive state, different patterns of EEGs may be extracted for each user/for each user's state. Therefore, when the user's EEGs are simply extracted and the extracted EEGs are mapped and analyzed with certain data, the accuracy may be deteriorated to identify and distinguish a current stress state of the user. Accordingly, the present invention performs a calibration method for EEGs for each user to accurately measure a cognitive state of the user based on the EEGs. A more detailed operation for the EEG sensing module 110 will be described below.

However, in the case of biosignals such as EEG, ECG, or the like, both a pattern (characteristic) and a level may vary for each user. For example, in the case of User A, in extracted Characteristic 1, there is the highest relation with stress and a level thereof may be changed in the range of 1 to 10, but in the case of User B, in extracted Characteristic 2 or 3, there may be the highest relation with stress and Characteristics 1 and 2 may not have the same range of scale, and thus the level thereof may vary for each Characteristic.

In addition, the range of levels may vary for each user's state, and the characteristics are mainly the same, but the range of levels mostly varies. That is, in the case of User A, when it is confirmed that Characteristic 1 best reflects the stress degree of the corresponding user, according to the user's state, in any case, the stress is measured in the range of 1 to 5, but in any case, the stress may be measured to levels of 15 to 20.

Accordingly, according to the present invention, calibration and normalization are performed to solve the problem that there may be a difference in stress level for each user and for each user's state. The detailed contents of calibration and normalization will be described below.

The ECG sensing module 130 may measure an electrocardiogram (ECG) using heart rate variability (HRV). Here, the electrocardiogram (ECG) is to express sequential electrical signals with heart rates as a graph, and as illustrated in FIG. 4, three wavelengths are formed on the electrocardiogram and include main characteristic points of P, Q, R, S, and T. At this time, P means atrial contraction, QRS means an electrical activity that causes ventricular contraction, and T means a waveform when the ventricles are depolarized and then repolarized.

Further, the heart rate variability (HRV) means an index representing how intervals of R peaks (or QRS complex) as peaks of the heart rate are changed. That is, the HRV may be confirmed as an RR interval or NN interval value between normal beats. The specific details thereof will be described below.

As illustrated in FIG. 1, the ECG sensing module 130 may also be included in the HMD device 100 in the center of the forehead with the EEG, and in some cases, may be attached near the chest, and in some cases, may also be attached to the wrist.

Generally, a stress measurement device using an ECG measured an ECG by measuring a potential difference between reference electrodes which become a reference in a measurement electrode from the measurement electrode attached near the chest, and used a variation degree of a RR interval value on the QRS graph. More specifically, in the process of controlling the heart rate by an autonomic nervous system, since the biosignals are expressed as the RR interval, a change of the RR interval is increased to have an irregular aspect as the activity degree (stress degree) of a sympathetic/parasympathetic nervous system constituting the autonomic nervous system is changed. This characteristic may be used as an indicator that reflects the stress state.

On the contrary, the ECG sensing module 130 of the present invention measures a potential difference between a reference electrode (REF electrode) attached to the center of the forehead with the EEG and a measurement electrode attached behind a remote controller as a VR controller to measure the ECG, that is, represented in the head and the hand when the user holds the measurement electrode by the hand, thereby measuring ECG data. Accordingly, it can be seen that an ECG sensing method of the present invention is the same in measurement principle as existing ECG sensing methods, but different in an analysis method. A more detailed operation for the ECG sensing module 130 will be described below.

The eye sensing module 120 may track the user's eyes using the eye sensor. The eye sensing module 120 may be provided in the HMD device 100 to be positioned around the user's eyes, especially under the eyes in order to track the user's eye (the motion of pupils) in real time.

The eye sensing module 120 is a light emitting device that emits light and a camera sensor for receiving (or sensing) light emitted from the light emitting device. More specifically, the eye sensing module 120 may photograph the light reflected from the user's eyes with a camera sensor, and transmit the photographed image to a processor.

The calibration module may calibrate biometric data to present a reference required for data analysis to be acquired thereafter using the EEG sensing module 110, the ECG sensing module 130, and the eye sensing module 120. More specifically, the calibration module 140 may acquire biometric data while the user is comfortable during a certain time (e.g., several seconds or minutes). For example, the calibration module 140 may perform biometric data calibration based on sounds, images or videos output through the output module 150 of the HMD device 100 while the user wears the HMD device 100. A detailed operation of the calibration module 140 will be described below.

The output module 150 may output result information on the biometric data sensed from the EEG sensing module 110, the ECG sensing module 130, and the eye sensing module 120 to sounds, images or videos. More specifically, the output module 150 may output texts, moving pictures, still images, a panoramic screen, VR images, augmented reality (AR) images, a speaker, a headset, or other various audiovisual information including the same, which may be output from a self-screen of the HMD device 100 or a display unit detached to the HMD device 100.

As described above, according to the present invention, only the sensor is attached to the HMD device to measure EEGs, an electrocardiogram, an electromyogram, eyes, and the like of the user at the same time without using an expensive medical device, thereby reducing cost burdens.

Further, the biosignal sensors are attached to an upper side of the HMD device 100, that is, near the forehead of the user to reduce an error occurrence of the sensors, and only the sensor is attached to the commonly used HMD device to measure the biometric data, thereby reducing a difficulty due to installation.

Configuration of Mental Care Server

Referring to FIG. 3, a mental care server 200 may include a communication module 240, a signal processing module 210, a diagnostic module 220, a learning module 230, a control module 250, and an output module 260. The mental care server 200 receives biosignals sensed from the HMD device 100 to analyze an EEG response, an ECG response, and an eye response.

The communication module 240 may transmit biosignals received from the EEG sensing module 110, the eye sensing module 120, and the ECG sensing module 130 to the signal processing module 210. In addition, the communication module 240 may be serial communication such as SPI, I2C, UART, etc. or may be wireless communication such as WiFi, Bluetooth, etc. according to physical positions of the eye sensing module 120 and the ECG sensing module 130.

A method for analyzing stress and managing individual mental health based on the biosignals received through the communication module 240 is as illustrated in FIG. 5.

Calibration Method Based on Received Biosignals

The mental care server 200 calibrates biosignals sensed from the EEG sensing module 110, the ECG sensing module 130, and the eye sensing module 120 (S510).

As illustrated in FIG. 8A, the calibration module 140 is a module which calibrates biosignals including EEGs, an ECG, an electromyogram, eyes, and the like received through the communication module 240 if necessary, and serves to generate standard stress information including information on a stress level or concentration. Here, the generating of the standard stress information means generating a reference required for analysis of result data to be obtained through the diagnostic module 220 or the learning module 230 based on the sensed biosignals. That is, the standard stress information may also mean a user's stress initial index (or value) before the user measures and analyzes the stress, or may mean a reference value for a specific emotion of the user. For example, when a step of ‘closing user's eyes and taking a rest for 1 minute’ is performed before the user measures and analyzes the stress, characteristics extracted from the data in this state are defined as a resting state. Thereafter, information on a specific emotion may be inferred through comparison how much a characteristic of biometric data obtained in the process of performing measurement contents or analysis contents is different from or similar to the resting state.

In other words, the mental care server 200 is a module which analyzes a change in stress level or concentration of the user viewing a VR content based on the standard stress information generated by the calibration module 140 to present a reference so as to infer information on specific emotion of the user. Here, the VR content means a content output in the form of images, videos, or sounds to the user through the output module 150 of the HMD device 100 to measure and analyze the stress level or concentration of the users and may be provided differently according to the stress level or concentration of the user.

The calibration module 140 may operate by dividing a case of calibrating an electromyogram (EMG) and an electrocardiogram (ECG) as data similar to EEGs and a case of calibrating eye data, respectively.

When a calibration object of the calibration module 140 is the EMG and the ECG as data similar to EEGs, the calibration module 140 may be used for a method which acquires biometric data by measuring electrical signals that occur in the brain, the skeletal muscle, or the heart, and then utilizes the acquired biometric data as stress standard information to find future content and to classify the emotions. For example, the EEGs of the biometric data may be classified into delta (δ) waves, theta (θ) waves, alpha (α) waves, beta (β) waves and gamma (g) waves according to a frequency range, and among the waves, the alpha (α) waves are mainly represented in a comfortable state such as relaxation, and the beta (β) waves are mainly represented in a tension or uneasy state.

Accordingly, when a ratio of the alpha (α) waves and the beta (β) waves of the user's EEGs measured while the user is in a comfortable state for several seconds is referred to as standard stress information, it may be determined that the user receives stimulation (stress) from the VR content when the ratio of the alpha (α) waves and the beta (β) waves measured when performing a stress measurement content step/stress analysis content step is higher than the standard stress information.

Also, when the calibration object of the calibration module 140 is the eye data, the calibration module 140 may be used for a method which collects the eye data of the user viewing the VR content and then uses the collected eye data as standard stress information to predict the eye of the user later. For example, when the eye data of the user gazing a white cross on a black screen for some seconds or viewing a video capable of improving the concentration is referred to as standard stress information, it is possible to determine stress and predict the eye data by analyzing the eye data measured when performing the stress measurement content step/stress analysis content step. A detailed operation for the prediction will be described below.

On the other hand, in some cases, a calibration operation by the calibration module 140 may also be omitted by the learning module 230 to be described below. In other words, in the present invention, when the stress is analyzed based on the standard stress information of the calibration module 140, since the user's stress may be analyzed only by the learning by the learning module 230, the calibration step may be omitted. In other words, when the characteristics of the user's stress index are repeatedly extracted by the learning module 230 and the stress index is leveled according to these characteristics, the calibration step may be omitted.

Method for Performing Stress Measurement Content

As illustrated in FIGS. 8B and 8C, the signal processing module 210 receives the standard stress information or the biometric data sensed from the biosignal sensors and then performs a stress measurement content (S520). Here, the performing of the stress measurement content means measuring the user's stress by providing a VR content to the user for stress diagnosis, measuring biosignals while the user views the VR content, and then providing a stress guiding screen. At this time, the stress guiding screen means a questionnaire test provided through the output module 150 of the HMD device 100 so as to diagnose the user's stress and may include at least one question and a plurality of answer items for the corresponding question. That is, it is preferable that the stress guiding screen is understood to be a questionnaire for the question and answer to be provided to the user in order to analyze the psychological factors of stress after measuring the stress.

While the user views the VR content or performs the question and answer on the stress guiding screen, the signal processing module 210 may measure biometric data such as an electroencephalogram (EEG), an electromyogram (EMG), an electrocardiogram (ECG), eyes, a photoplethysmography (PPG), etc. Here, since the electrocardiogram (ECG) is measured by using heart rate variability (HRV), which is an interval of R Peak (or QRS Complex) representing a peak of the heart rate, the heart rate variability (HRV) may be confirmed as an RR interval value. In addition, a low frequency area and a high frequency area between RR intervals or complexity, uniformity, etc. of the interval mean the balance and stress range of the autonomic nervous system.

More specifically, in the process of controlling the heart rate by an autonomic nervous system, since the biosignals are expressed as the RR interval, a change in the RR interval may occur as the activity degree (stress degree) of a sympathetic/parasympathetic nervous system constituting the autonomic nervous system is changed. For example, if the stress increases, a change of RR interval is reduced to show a regular aspect, but when the stress is alleviated, the change of the RR interval is increased to show an irregular aspect.

Thus, the signal processing module 210 extracts these characteristics from the biometric data measured while the user is viewing the VR content (S610), and then the diagnostic module 220 may be used as an indicator of diagnosing the user's stress state based on the extracted characteristics. Here, as illustrated in FIGS. 8B and 8C, the characteristics mean the eye response, the EEG response, the ECG response, etc. extracted by the signal processing module 210 through the question-and-answer process to the plurality of questions displayed on the stress guiding screen of the user, and a method of extracting them will be described later.

First, the signal processing module 210 may perform a basic questionnaire test, as illustrated in FIG. 8B, to detect a baseline of a reading pattern that reads the stress guiding screen by the user. That is, by first detecting the baseline before this questionnaire test provided through the stress guiding screen, it is possible to present a reference for analyzing a reading pattern for the questionnaire test as illustrated in FIG. 8C. Therefore, as illustrated in FIG. 8B, in the basic questionnaire test step, questions for the basic questionnaire test such as questions capable of simply recognizing the fact, ambiguous questions without correct answers, or emotionally stimulating questions may be complexly provided.

Hereinafter, a method of detecting a leading pattern from the eye response, the EEG response, the ECG response, and the heart rate variability (HRV) will be described in detail.

Detection of Eye Response

The eye response may be detected based on the extracted eye pattern using various data used in the eye motion. For example, the data used in the eye motion may be defined as data such as eye fixation in which the eyes stay in one point at a moment, saccade which is rapid movement of eyes, a scan path as an eye path, and revisit in which the eyes return to a specific point for detection of detailed characteristics.

Meanwhile, how many the eye fixation data are formed when the user makes the question and answer for the questionnaire test may mean a load degree of a visual perception process of the corresponding question.

Further, through complexity of the eye fixation data or a variability of the eye pattern between specific answer alternatives (Do not, Do, Yes, No, etc.) of the user, it may be confirmed how confident the user has answered this question.

Further, the eye fixation and saccade data of the user are analyzed to measure the sincerity of the user for the question. For example, whether the user has read all of the questions, whether the user makes the answer all of the answer items with thinking carefully, and the like may be measured.

Detection of EEG Response

The EEG response may be detected based on the extracted EEG pattern using a potential of an EEG specific region. For example, after the question is given to the user, how the user is familiar to the corresponding question or whether there has been an emotional change may be confirmed through a potential change p300 of the EEG specific region responding within 300 ms in the EEG of the user. For example, when pictures unfamiliar with the user and pictures familiar with the user are randomly arranged to be exposed at a very short time, event-related potential (ERP) stimulation may be shown to be larger when the user views the familiar pictures. Accordingly, according to the present invention, a pattern of the actual ERP stimulation may be predicted through the random arrangement to be used as a synchronization time based on the predicted ERP stimulation pattern. Further, in the present invention, the familiar pictures may be images which are tagged, that is, may be tagged for pictures that have been repeatedly exposed to users, and may be images predicted to have a lot of actual exposure to people, for example, a window wallpaper, etc. Here, the system for analyzing the stress and managing the individual mental health using the HMD device of the present invention may further include a matrix computing module for deep learning and may locally perform a computation more efficiently by tagging based on the matrix computing module.

In other words, the time of the system may be calibrated so that a time (mobile time) at which stimulation is given and a time (time of the EEG sensor) when ERP stimulation appears are the same as each other. The specific details thereof will be described below.

In addition, a question with a response and a question without a response of the EEG response of the user are differentially analyzed to measure the emotional stability of the user received by the question. For example, if there is no response of P300 (a potential change of the EEG specific region responding within 300 ms in the EEG of the user), this may recognize that there is no emotional and unconscious influence on this question.

Further, in the EEG occurring when the user is reading the taxt, when the EEG power in a region of beta ((3) waves/gamma (g) waves is excessively higher than that of when the user is reading a basic present questionnaire test of the baseline questions, it may be analyzed that cognitive/emotional stress has occurred for these questions.

Detection of ECG Response

The ECG response may be detected through an ECG change occurring while the user performs the questionnaire test through the stress guiding screen. At this time, the ECG response may generate additional information based on various ECG change conditions. Here, the ECG change conditions mean heart rate variability, a complexity change in heart rate, an abnormal phenomenon of a heart pattern, etc.

When the ECG change condition is the heart rate variability, while the user is reading the present questionnaire test during the basic questionnaire test, it may be recognized that the user has been emotionally changed in this questionnaire item through an interval that heart rate changes instantaneously.

When the ECG change condition is the complexity change in the heart rate, a change in complexity of heart rate means a stress strength, through that the user's complexity was complicated in a specific questionnaire item, it may be recognized that the user has received the stress emotionally and perceivably in this questionnaire item.

In addition, when the ECG change condition is an abnormal phenomenon of the heart pattern, it may be recognized that an unspecified abnormal response of the heart pattern, such as atrial fibrillation, is a user's health fatal problem. This can be linked with health-specific diseases (heart attacks, hypertension), and the like, and may be linked to diagnosis and screening (or screening inspection) for the related diseases later.

At this time, the present invention may selectively apply accuracy enhancement conditions to increase the analysis accuracy of the questionnaire test. Here, the accuracy enhancement condition means an order change of answer items, a position change of the answer item, an order change of the questionnaire, and the like.

More specifically, when the order of the answer items is changed to increase the analysis accuracy, the order of answer items such as Do, Do not/Yes, No, etc. is randomly changed to more accurately analyze the eye pattern of the user. In other words, the reason for randomly changing the order of answer items is to investigate the response to whether to habitually see the question-and-answer items of the following items when the user answers, and may be facilitated to observe the sincerity and cognitive load process in the user's visual perception information processing process through the user's eye pattern, and the like.

In addition, to increase the analysis accuracy, a location of answer item is changed to determine whether the user's response to the corresponding item is constant or whether the user sincerely reads the question of the item, etc., and the order of the questionnaire is changed to improve the accuracy of the analysis method.

Method for Performing Stress Analysis Content

Next, the stress analysis result content is performed according to the result of performing the stress measurement content (S530). Here, the performing of the stress analysis result content means analyzing the stress by various methods from the extracted characteristics. In the present invention, the method of performing the stress analysis result content may be largely divided into three methods.

First, after the signal processing module 210 extracts the characteristics from the biometric data, the diagnostic module 220 substitutes the extracted characteristics to the stress level (S620). In other words, the diagnostic module 220 may diagnose the user's stress by substituting the extracted characteristics during the user's questionnaire test to the stress level.

At this time, the stress level may be calculated using the following Equation 1.

Stress level=EG1*Weeg1 . . . + . . . EEGN*WeegN+EGG1*Wecg1 . . . + . . . ECGM*WecgM+EYE1*Weye1 . . . + . . . EYEK*WeyeK [Equation 1]

Here, W represents a weight value of each sensor and is determined by experimental data of an individual user (subject).

At this time, the weight value W may be selected as accuracy measured by an individual sensor method (modality). In other words, when the accuracy of the ECG is 80%, the accuracy of EEG is 70%, and the accuracy of the eye data is 50%, the corresponding values are normalized to set the weight value W as 0.4, 0.35, and 0.25. However, when there are many experimental data, the weight value W may be determined by learning through learning module 230. In other words, if the user's stress level is already known from the response of the questionnaire, the weight value W may be determined through a simple linear regression method.

In a general stress level measuring system, since an EEG sensor, an ECG sensor, and an eye sensor have different measurement sensors, different characteristics may be extracted from each sensor or the characteristics may be learned and extracted using deep learning. At this time, in the general stress level measuring system, various characteristics may be extracted from raw data acquired from the sensor by any method and all of the characteristics are used.

On the contrary, there is a difference from the existing technology in that the system for analyzing the stress and managing the individual mental health according to an embodiment of the present invention may extract very various different characteristics in addition to information clearly known on the stress level and select a weight value capable of best confirming the stress level through learning. Accordingly, in the case of the present invention, since the characteristic dimension of the learned data is very large, the learning may be difficult, so that it is necessary to be a very sophisticated learning model (machine learning, deep learning model).

Here, according to the present invention, it is possible to design a model of predicting the stress and calculate a stress index based on the learned model after selecting a characteristic with the highest association with the stress level by learning using a recurrent neural network (RNN) or a long short term memory (LSTM).

Further, the diagnostic module 220 may also predict the stress by comparing a change in stress measurement information with standard stress information generated by the calibration module 140. Here, the stress measurement information means information including a stress index, concentration, sincerity, etc. measured from the user viewing the VR content and the stress guiding screen after performing the calibration step.

Method for Performing Stress Relaxation Content

Thereafter, when the stress analysis result is significantly higher than a predetermined stress level, a relaxation content according to the stress analysis result is performed (S540). More specifically, the output module 260 may output a content including various information according to the stress analysis result as a result screen (S630). For example, the relaxation content may include sounds, images, or videos as the content provided to lower the stress index of the user.

Also, the relaxation content may be output differently by the user or by the user's stress level.

The control module 250 may control the signal processing module 210, the diagnostic module 220, the learning module 230, and the output module 260.

Time Synchronization Method for a Series of Signals

Further, in order to analyze signals such as EEG, eyes, ECG, safety, EMG, etc. as various biosignals sensed by the HMD device 100, changes in EEG, eyes, ECG, safety, EMG, etc. of the user are measured for a very short time of at least 300 ms or less. In this case, a clock time of the HMD device 100 for displaying the stress guiding screen and a clock time of the biometric sensor that acquires biometric information of the user may be different from each other, or a clock time of the biometric sensor and a clock time of a processor of analyzing the biometric information may be different from each other.

As a result, the system for analyzing the stress and managing the individual mental health may perform time synchronizing for a series of signals by using at least two or more synchronization sensing signals so as to correctly analyze a change in biometric information according to user's video viewing.

Specifically, the mental care server 200 of the present invention receives a first synchronization sensing signal related to a first sensing signal (EEG sensing signal) received from a first biosignal sensor and receives a second synchronization sensing signal related to a second sensing signal (ECG sensing signal) received from a second biosignal sensor. Although described below, in the present invention, preferably, it will be understood that an event trigger signal is expressed based on the first synchronization sensing signal and the second synchronization sensing signal.

Here, the first synchronization sensing signal and the second synchronization sensing signal may be associated with at least two series of signals, respectively. For example, a series of signals may include at least one of an EEG sensing signal, an ECG sensing signal, a virtual reality image or a video signal, or various signals in the system.

Further, the first biosignal sensor and the second biosignal sensor may be at least one of a motion sensor that outputs a synchronization sensing signal representing motion information of the user, an illumination sensor that outputs a synchronization sensing signal representing ambient brightness information, an optical sensor that outputs a synchronization sensing signal representing light information of a predetermined light amount, and a sonic sensor that outputs a synchronization sensing signal representing predetermined audio information.

Further, the mental care server 200 receives a first synchronization sensing signal induced from the event trigger signal and received from the first biosignal sensor, receives a second synchronization sensing signal induced from the event trigger signal and received from the second biosignal sensor, calculates time difference information of the first synchronization sensing signal and the second synchronization sensing signal based on a time when the event trigger signal is expressed, and may synchronize the first biosignal sensor and the second biosignal sensor based on the time difference information. For example, the event trigger signal is a signal generated when the stimulation is given to the user, and preferably understood as a signal generated when the familiar/unfamiliar pictures to the user are randomly exposed or a short sound (beep) of a high-sound band is given as acoustic stimulation.

In other words, the event trigger signal may be displayed on a display of the HMD device 100 by randomly arranging the familiar pictures and the unfamiliar pictures, and may be a high-sound band (beep) having a range of about 10 to 90 db when a range of the general stimulation is at most about 500 to 10,000 hz, and may be a blinking screen displayed on the display of the HMD device 100.

Hereinafter, when the event trigger signal is detected, it will be described in two cases.

First, in the present invention, when the EEG sensing signal by the visual stimulation is detected, the time of the system may be calibrated so that a time expressing the event trigger signal (time when the stimulation is actually given) and a time when the ERP stimulation appears (time of the EEG sensor, that is, time when the event-related potential is measured) are the same as each other.

According to the present invention, after the event trigger signal is expressed, a time when the event trigger signal is expressed within a predetermined time and a time when the ERP stimulation (the first synchronization signal or the second synchronization signal) appears are measured, respectively. Thereafter, when the two measured signals are different from each other, the time may be calibrated so that a difference in the two times becomes the same. For example, since the event-related potentials (ERPs) when seeing the familiar picture by randomly exposing a familiar picture and an unfamiliar picture to the user are different from each other, time differences corresponding to the ERPs for the familiar picture and the unfamiliar picture are measured, respectively, to calibrate the time. However, the ERPs when seeing the familiar picture and the unfamiliar picture should have a difference of a predetermined level or more, but there may be no difference. Therefore, in this case, the accuracy of the measurement may be increased by re-combining the random arrangement of familiar pictures and unfamiliar pictures.

In addition, the present invention may calibrate the time based on a predicted ERP stimulation pattern by randomly exposing the familiar pictures and unfamiliar pictures to the user.

Generally, when the visual stimulation of a specific frequency region on the screen is exposed to the user, there is a phenomenon that the user's EEG is synchronized in accordance with the corresponding frequency. That is, the user's EEG may be synchronized in accordance with the corresponding frequency. Accordingly, in the case where it is assumed that the corresponding region is viewed by the user when any portion of the screen, for example, a blinking screen with 60 Hz is displayed (at a level that the user is not recognized), on the system of the present invention, whether the user views the region thereof may be confirmed. The time of the system may be calibrated so that the EEG synchronization time (EEG sensor time) and a time (mobile time) when the content is reproduced are the same as each other.

Further, according to the present invention, in the case of detecting the EEG sensing signal by audio stimulation, when a short sound (beep) of a high-sound band is given as auditory stimulation, the time synchronization may be performed by using that the EEG response to the corresponding stimulation is immediately shown. However, even if the time synchronization is accurately matched, in the case of synchronization between internal sensor systems, an error according to the time is rarely caused, but in the case of synchronization between mobile or third devices to which the content is reproduced, a delay error may also occur according to a network state. Accordingly, the present invention may confirm a time synchronization error by exposing a signal detection method optionally in the middle of the content, and perform the time calibration.

Therefore, a HDM device 900 according to another embodiment of the present invention performs time synchronization for a series of signals using two synchronization sensors to calibrate a time error between components in the system and a time error between different systems, thereby improving the accuracy of the measurement.

Although the exemplary embodiments of the present invention have been described in detail with reference to the accompanying drawings, the present invention is not limited thereto and may be embodied in many different forms without departing from the technical concept of the present invention. Therefore, the exemplary embodiments disclosed in the present invention are intended not to limit the technical spirit of the present invention but to describe the present invention and the scope of the technical spirit of the present invention is not limited by these exemplary embodiments. Therefore, it should be understood that the above-described exemplary embodiments are illustrative in all aspects and do not limit the present invention. The protective scope of the present invention should be construed based on the appended claims, and all the technical spirits in the equivalent scope thereof should be construed as falling within the scope of the present invention.

Configuration of HMD Device of System for Analyzing Stress and Managing Individual Mental Health

FIG. 1 is an overall schematic diagram of a system for analyzing stress and managing individual mental health using an HMD device according to an embodiment of the present invention. FIG. 2 is a block diagram for describing a HMD device according to an embodiment of the present invention. FIG. 3 is a block diagram for describing a mental care server according to an embodiment of the present invention. FIG. 4 is an exemplary graph for describing an electrocardiogram (ECG) according to an embodiment of the present invention. FIG. 5 is an overall flowchart for describing a method for analyzing stress and managing individual mental health according to an embodiment of the present invention. FIG. 6 is a flowchart for describing a method for performing an analysis content of a mental care server according to an embodiment of the present invention. FIG. 7 is a diagram for describing a plurality of biosignal sensors attached to an HMD device according to an embodiment of the present invention. FIGS. 8A to 8C are exemplary diagrams for describing a stress guiding screen according to an embodiment of the present invention.

Referring to FIG. 1, a system for analyzing stress and managing individual mental health includes a HMD device 100, biosignal sensors, and a mental care server 200.

The HMD device 100 includes biosignal sensors as various types of wearable devices that may be worn by a user and may sense biosignals of the user. Here, the biosignals may mean various signals that are generated from the user's body such as EEGs, eyes, motion of pupils, heart rate, and blood pressure of the user.

In the present invention, the HMD device 100 may be mounted on the head as a head-mounted display (HMD) to directly/indirectly present images to the user.

For example, the HMD device 100 may be a device in the form of supporting virtual reality including a display unit by itself such as Oculus® VR (virtual reality) and may also be a device in a similar form to Gear® VR used by mounting the display unit on the HMD mount. Alternatively, the HMD device 100 may be a device that supports augmented reality (AR) in the form of Google Glass® or Microsoft HoloLens®. Alternatively, the HMD device 100 may be a device that supports mixed reality such as Windows mixed reality (MR) or Odyssey Plus MR.

As illustrated in FIG. 2, the HMD device 100 may include an EEG sensing module 110 measuring an electroencephalogram from an electroencephalogram (EEG) sensor, an eye sensing module 120 measuring the motion of pupils from an eye sensor, an ECG sensing module 130 measuring an electrocardiogram from an electrocardiogram (ECG) sensor, a calibration module 140, and an output module 150. Meanwhile, in the present invention, as long as the EEG sensor, the eye sensor, and the ECG sensor may be easily in contact with a body site so as to measure biosignals of the user, the sensors are not limited only to the HMD device 100, but may be any type of wearable devices. For example, the HMD device 100 may be a headset, a smart watch, an earphone, a mobile device, etc.

As illustrated in FIG. 7, the biosignal sensors are attached to the HMD device 100 and include an ECG sensor 101, an EEG sensor 102, and an eye sensor 103.

The EEG sensing module 110 may sense the electroencephalogram of the user wearing the HMD device 100. The EEG sensing module 110 may include at least one electroencephalogram (EEG) sensor. When the user wears the HMD device, the EEG sensor attached to the HMD device 100 is in contact with a body site, such as a head or forehead where the EEG of the user can be measured, and then the EEG sensing module 110 may measure the EEG of the user. The EEG sensing module 110 may measure various frequencies of EEGs generated from the contacted body site of the user or electrical/optical frequencies changed according to an activation state of the brain.

However, since the EEGs are biosignals, there may be a difference according to a peripheral situation or an inner body situation of the user for each user or even in the same user. Thus, even in the same cognitive state, different patterns of EEGs may be extracted for each user/for each user's state. Therefore, when the user's EEGs are simply extracted and the extracted EEGs are mapped and analyzed with cetain data, the accuracy may be deteriorated to identify and distinguish a current stress state of the user. Accordingly, the present invention performs a calibration method for EEGs for each user to accurately measure a cognitive state of the user based on the EEGs. A more detailed operation for the EEG sensing module 110 will be described below.

However, in the case of biosignals such as EEG, ECG, or the like, both a pattern (characteristic) and a level may vary for each user. For example, in the case of User A, in extracted Characteristic 1, there is the highest relation with stress and a level thereof may be changed in the range of 1 to 10, but in the case of User B, in extracted Characteristic 2 or 3, there may be the highest relation with stress and Characteristics 1 and 2 may not have the same range of scale, and thus the level thereof may vary for each Characteristic.

In addition, the range of levels may vary for each user's state, and the characteristics are mainly the same, but the range of levels mostly varies. That is, in the case of User A, when it is confirmed that Characteristic 1 best reflects the stress degree of the corresponding user, according to the user's state, in any case, the stress is measured in the range of 1 to 5, but in any case, the stress may be measured to levels of 15 to 20.

Accordingly, according to the present invention, calibration and normalization are performed to solve the problem that there may be a difference in stress level for each user and for each user's state. The detailed contents of calibration and normalization will be described below.

The ECG sensing module 130 may measure an electrocardiogram (ECG) using heart rate variability (HRV). Here, the electrocardiogram (ECG) is to express sequential electrical signals with heart rates as a graph, and as illustrated in FIG. 4, three wavelengths are formed on the electrocardiogram and include main characteristic points of P, Q, R, S, and T. At this time, P means atrial contraction, QRS means electrical activity that causes ventricular contraction, and T means a waveform when the ventricles are depolarized and then repolarized.

Further, the heart rate variability (HRV) means an index representing how intervals of R peaks (or QRS complex) as peaks of the heart rate are changed. That is, the HRV may be confirmed as an RR interval or NN interval value between normal beats. The specific details thereof will be described below.

As illustrated in FIG. 1, the ECG sensing module 130 may also be included in the HMD device 100 in the center of the forehead with the EEG, and in some cases, may be attached near the chest, and in some cases, may also be attached to the wrist.

Generally, a stress measurement device using an ECG measured an ECG by measuring a potential difference between reference electrodes which become a reference in a measurement electrode from the measurement electrode attached near the chest, and used a variation degree of a RR interval value on the QRS graph. More specifically, in the process of controlling the heart rate by an autonomic nervous system, since the biosignals are expressed as the RR interval, a change of the RR interval is increased to have an irregular aspect as the activity degree (stress degree) of a sympathetic/parasympathetic nervous system constituting the autonomic nervous system is changed. This characteristic may be used as an indicator that reflects the stress state.

On the contrary, the ECG sensing module 130 of the present invention measures a potential difference between a reference electrode (REF electrode) attached to the center of the forehead with the EEG and a measurement electrode attached behind a remote controller as a VR controller to measure the ECG, that is, represented in the head and the hand when the user holds the measurement electrode by the hand, thereby measuring ECG data. Accordingly, it can be seen that an ECG sensing method of the present invention is the same in measurement principle as existing ECG sensing methods, but different in an analysis method. A more detailed operation for the ECG sensing module 130 will be described below.

The eye sensing module 120 may track the user's eyes using the eye sensor. The eye sensing module 120 may be provided in the HMD device 100 to be positioned around the user's eyes, especially under the eyes in order to track the user's eye (the motion of pupils) in real time.

The eye sensing module 120 is a light emitting device that emits light and a camera sensor for receiving (or sensing) light emitted from the light emitting device. More specifically, the eye sensing module 120 may photograph the light reflected from the user's eyes with a camera sensor, and transmit the photographed image to a processor.

The calibration module may calibrate biometric data to present a reference required for data analysis to be acquired thereafter using the EEG sensing module 110, the ECG sensing module 130, and the eye sensing module 120. More specifically, the calibration module 140 may acquire biometric data while the user is comfortable during a certain time (e.g., several seconds or minutes). For example, the calibration module 140 may perform biometric data calibration based on sounds, images or videos output through the output module 150 of the HMD device 100 while the user wears the HMD device 100. A detailed operation of the calibration module 140 will be described below.

The output module 150 may output result information on the biometric data sensed from the EEG sensing module 110, the ECG sensing module 130, and the eye sensing module 120 to sounds, images or videos. More specifically, the output module 150 may output texts, moving pictures, still images, a panoramic screen, VR images, augmented reality (AR) images, a speaker, a headset, or other various audiovisual information including the same, which may be output from a self-screen of the HMD device 100 or a display unit detached to the HMD device 100.

As described above, according to the present invention, only the sensor is attached to the HMD device to measure EEGs, an electrocardiogram, an electromyogram, eyes, and the like of the user at the same time without using an expensive medical device, thereby reducing cost burdens.

Further, the biosignal sensors are attached to an upper side of the HMD device 100, that is, near the forehead of the user to reduce an error occurrence of the sensors, and only the sensor is attached to the commonly used HMD device to measure the biometric data, thereby reducing a difficulty due to installation.

Configuration of Mental Care Server

Referring to FIG. 3, a mental care server 200 may include a communication module 240, a signal processing module 210, a diagnostic module 220, a learning module 230, a control module 250, and an output module 260. The mental care server 200 receives biosignals sensed from the HMD device 100 to analyze an EEG response, an ECG response, and an eye response.

The communication module 240 may transmit biosignals received from the EEG sensing module 110, the eye sensing module 120, and the ECG sensing module 130 to the signal processing module 210. In addition, the communication module 240 may be serial communication such as SPI, I2C, UART, etc. or may be wireless communication such as WiFi, Bluetooth, etc. according to physical positions of the eye sensing module 120 and the ECG sensing module 130.

A method for analyzing stress and managing individual mental health based on the biosignals received through the communication module 240 is as illustrated in FIG. 5.

Calibration Method Based on Received Biosignals

The mental care server 200 calibrates biosignals sensed from the EEG sensing module 110, the ECG sensing module 130, and the eye sensing module 120 (S510).

As illustrated in FIG. 8A, the calibration module 140 is a module which calibrates biosignals including EEGs, an ECG, an electromyogram, eyes, and the like received through the communication module 240 if necessary, and serves to generate standard stress information including information on a stress level or concentration. Here, the generating of the standard stress information means generating a reference required for analysis of result data to be obtained through the diagnostic module 220 or the learning module 230 based on the sensed biosignals. That is, the standard stress information may also mean a user's stress initial index (or value) before the user measures and analyzes the stress, or may mean a reference value for a specific emotion of the user. For example, when a step of ‘closing user's eyes and taking a rest for 1 minute’ is performed before the user measures and analyzes the stress, characteristics extracted from the data in this state are defined as a resting state. Thereafter, information on a specific emotion may be inferred through comparison how much a characteristic of biometric data obtained in the process of performing measurement contents or analysis contents is different from or similar to the resting state.

In other words, the mental care server 200 is a module which analyzes a change in stress level or concentration of the user viewing a VR content based on the standard stress information generated by the calibration module 140 to present a reference so as to infer information on specific emotion of the user. Here, the VR content means a content output in the form of images, videos, or sounds to the user through the output module 150 of the HMD device 100 to measure and analyze the stress level or concentration of the users and may be provided differently according to the stress level or concentration of the user.

The calibration module 140 may operate by dividing a case of calibrating an electromyogram (EMG) and an electrocardiogram (ECG) as data similar to EEGs and a case of calibrating eye data, respectively.

When a calibration object of the calibration module 140 is the EMG and the ECG as data similar to EEGs, the calibration module 140 may be used for a method which acquires biometric data by measuring electrical signals that occur in the brain, the skeletal muscle, or the heart, and then utilizes the acquired biometric data as stress standard information to find future content and to classify the emotions. For example, the EEGs of the biometric data may be classified into delta (δ) waves, theta (θ) waves, alpha (α) waves, beta (β) waves and gamma (g) waves according to a frequency range, and among the waves, the alpha (α) waves are mainly represented in a comfortable state such as relaxation, and the beta (β) waves are mainly represented in a tension or uneasy state.

Accordingly, when a ratio of the alpha (α) waves and the beta (β) waves of the user's EEGs measured while the user is in a comfortable state for several seconds is referred to as standard stress information, it may be determined that the user receives stimulation (stress) from the VR content when the ratio of the alpha (α) waves and the beta (β) waves measured when performing a stress measurement content step/stress analysis content step is higher than the standard stress information.

Also, when the calibration object of the calibration module 140 is the eye data, the calibration module 140 may be used for a method which collects the eye data of the user viewing the VR content and then uses the collected eye data as standard stress information to predict the eye of the user later. For example, when the eye data of the user gazing a white cross on a black screen for some seconds or viewing a video capable of improving the concentration is referred to as standard stress information, it is possible to determine stress and predict the eye data by analyzing the eye data measured when performing the stress measurement content step/stress analysis content step. A detailed operation for the prediction will be described below.

On the other hand, in some cases, a calibration operation by the calibration module 140 may also be omitted by the learning module 230 to be described below. In other words, in the present invention, when the stress is analyzed based on the standard stress information of the calibration module 140, since the user's stress may be analyzed only by the learning by the learning module 230, the calibration step may be omitted. In other words, when the characteristics of the user's stress index are repeatedly extracted by the learning module 230 and the stress index is leveled according to these characteristics, the calibration step may be omitted.

Method for Performing Stress Measurement Content

As illustrated in FIGS. 8B and 8C, the signal processing module 210 receives the standard stress information or the biometric data sensed from the biosignal sensors and then performs a stress measurement content (S520). Here, the performing of the stress measurement content means measuring the user's stress by providing a VR content to the user for stress diagnosis, measuring biosignals while the user views the VR content, and then providing a stress guiding screen. At this time, the stress guiding screen means a questionnaire test provided through the output module 150 of the HMD device 100 so as to diagnose the user's stress and may include at least one question and a plurality of answer items for the corresponding question. That is, it is preferable that the stress guiding screen is understood to be a questionnaire for the question and answer to be provided to the user in order to analyze the psychological factors of stress after measuring the stress.

While the user views the VR content or performs the question and answer on the stress guiding screen, the signal processing module 210 may measure biometric data such as an electroencephalogram (EEG), an electromyogram (EMG), an electrocardiogram (ECG), eyes, a photoplethysmography (PPG), etc. Here, since the electrocardiogram (ECG) is measured by using heart rate variability (HRV), which is an interval of R Peak (or QRS Complex) representing a peak of the heart rate, the heart rate variability (HRV) may be confirmed as an RR interval value. In addition, a low frequency area and a high frequency area between RR intervals or complexity, uniformity, etc. of the interval mean the balance and stress range of the autonomic nervous system.

More specifically, in the process of controlling the heart rate by an autonomic nervous system, since the biosignals are expressed as the RR interval, a change in the RR interval may occur as the activity degree (stress degree) of a sympathetic/parasympathetic nervous system constituting the autonomic nervous system is changed. For example, if the stress increases, a change of RR interval is reduced to show a regular aspect, but when the stress is alleviated, the change of the RR interval is increased to show an irregular aspect.

Thus, the signal processing module 210 extracts these characteristics from the biometric data measured while the user is viewing the VR content (S610), and then the diagnostic module 220 may be used as an indicator of diagnosing the user's stress state based on the extracted characteristics. Here, as illustrated in FIGS. 8B and 8C, the characteristics mean the eye response, the EEG response, the ECG response, etc. extracted by the signal processing module 210 through the question-and-answer process to the plurality of questions displayed on the stress guiding screen of the user, and a method of extracting them will be described later.

First, the signal processing module 210 may perform a basic questionnaire test, as illustrated in FIG. 8B, to detect a baseline of a reading pattern that reads the stress guiding screen by the user. That is, by first detecting the baseline before this questionnaire test provided through the stress guiding screen, it is possible to present a reference for analyzing a reading pattern for the questionnaire test as illustrated in FIG. 8C. Therefore, as illustrated in FIG. 8B, in the basic questionnaire test step, questions for the basic questionnaire test such as questions capable of simply recognizing the fact, ambiguous questions without correct answers, or emotionally stimulating questions may be complexly provided.

Hereinafter, a method of detecting a leading pattern from the eye response, the EEG response, the ECG response, and the heart rate variability (HRV) will be described in detail.

Detection of Eye Response

The eye response may be detected based on the extracted eye pattern using various data used in the eye motion. For example, the data used in the eye motion may be defined as data such as eye fixation in which the eyes stay in one point at a moment, saccade which is rapid movement of eyes, a scan path as an eye path, and revisit in which the eyes return to a specific point for detection of detailed characteristics.

Meanwhile, how many the eye fixation data are formed when the user makes the question and answer for the questionnaire test may mean a load degree of a visual perception process of the corresponding question.

Further, through complexity of the eye fixation data or a variability of the eye pattern between specific answer alternatives (Do not, Do, Yes, No, etc.) of the user, it may be confirmed how confident the user has answered this question.

Further, the eye fixation and saccade data of the user are analyzed to measure the sincerity of the user for the question. For example, whether the user has read all of the questions, whether the user makes the answer all of the answer items with thinking carefully, and the like may be measured.

Detection of EEG Response

The EEG response may be detected based on the extracted EEG pattern using a potential of an EEG specific region. For example, after the question is given to the user, how the user is familiar to the corresponding question or whether there has been an emotional change may be confirmed through a potential change p300 of the EEG specific region responding within 300 ms in the EEG of the user. For example, when pictures unfamiliar with the user and pictures familiar with the user are randomly arranged to be exposed at a very short time, event-related potential (ERP) stimulation may be shown to be larger when the user views the familiar pictures. Accordingly, according to the present invention, a pattern of the actual ERP stimulation may be predicted through the random arrangement to be used as a synchronization time based on the predicted ERP stimulation pattern. Further, in the present invention, the familiar pictures may be images which are tagged, that is, may be tagged for pictures that have been repeatedly exposed to users, and may be images predicted to have a lot of actual exposure to people, for example, a window wallpaper, etc. Here, the system for analyzing the stress and managing the individual mental health using the HMD device of the present invention may further include a matrix computing module for deep learning and may locally perform a computation more efficiently by tagging based on the matrix computing module.

In other words, the time of the system may be calibrated so that a time (mobile time) at which stimulation is given and a time (time of the EEG sensor) when ERP stimulation appears are the same as each other. The specific details thereof will be described below.

In addition, a question with a response and a question without a response of the EEG response of the user are differentially analyzed to measure the emotional stability of the user received by the question. For example, if there is no response of P300 (a potential change of the EEG specific region responding within 300 ms in the EEG of the user), this may recognize that there is no emotional and unconscious influence on this question.

Further, in the EEG occurring when the user is reading the text, when the EEG power in a region of beta ((3) waves/gamma (g) waves is excessively higher than that of when the user is reading a basic present questionnaire test of the baseline questions, it may be analyzed that cognitive/emotional stress has occurred for these questions.

Detection of ECG Response

The ECG response may be detected through an ECG change occurring while the user performs the questionnaire test through the stress guiding screen. At this time, the ECG response may generate additional information based on various ECG change conditions. Here, the ECG change conditions mean heart rate variability, a complexity change in heart rate, an abnormal phenomenon of a heart pattern, etc.

When the ECG change condition is the heart rate variability, while the user is reading the present questionnaire test during the basic questionnaire test, it may be recognized that the user has been emotionally changed in this questionnaire item through an interval that heart rate changes instantaneously.

When the ECG change condition is the complexity change in the heart rate, a change in complexity of heart rate means a stress strength, through that the user's complexity was complicated in a specific questionnaire item, it may be recognized that the user has received the stress emotionally and perceivably in this questionnaire item.

In addition, when the ECG change condition is an abnormal phenomenon of the heart pattern, it may be recognized that an unspecified abnormal response of the heart pattern, such as atrial fibrillation, is a user's health fatal problem. This can be linked with health-specific diseases (heart attacks, hypertension), and the like, and may be linked to diagnosis and screening (or screening inspection) for the related diseases later.

At this time, the present invention may selectively apply accuracy enhancement conditions to increase the analysis accuracy of the questionnaire test. Here, the accuracy enhancement condition means an order change of answer items, a position change of the answer item, an order change of the questionnaire, and the like.

More specifically, when the order of the answer items is changed to increase the analysis accuracy, the order of answer items such as Do, Do not/Yes, No, etc. is randomly changed to more accurately analyze the eye pattern of the user. In other words, the reason for randomly changing the order of answer items is to investigate the response to whether to habitually see the question-and-answer items of the following items when the user answers, and may be facilitated to observe the sincerity and cognitive load process in the user's visual perception information processing process through the user's eye pattern, and the like.

In addition, to increase the analysis accuracy, a location of answer item is changed to determine whether the user's response to the corresponding item is constant or whether the user sincerely reads the question of the item, etc., and the order of the questionnaire is changed to improve the accuracy of the analysis method.

Method for Performing Stress Analysis Content

Next, the stress analysis result content is performed according to the result of performing the stress measurement content (S530). Here, the performing of the stress analysis result content means analyzing the stress by various methods from the extracted characteristics. In the present invention, the method of performing the stress analysis result content may be largely divided into three methods.

First, after the signal processing module 210 extracts the characteristics from the biometric data, the diagnostic module 220 substitutes the extracted characteristics to the stress level (S620). In other words, the diagnostic module 220 may diagnose the user's stress by substituting the extracted characteristics during the user's questionnaire test to the stress level.

At this time, the stress level may be calculated using the following Equation 1.

Stress level=EG1*Weeg1 . . . + . . . EEGN*WeegN+EGG1*Wecg1 . . . + . . . ECGM*WecgM+EYE1*Weye1 . . . + . . . EYEK*WeyeK [Equation 1]

Here, W represents a weight value of each sensor and is determined by experimental data of an individual user (subject).

At this time, the weight value W may be selected as accuracy measured by an individual sensor method (modality). In other words, when the accuracy of the ECG is 80%, the accuracy of EEG is 70%, and the accuracy of the eye data is 50%, the corresponding values are normalized to set the weight value W as 0.4, 0.35, and 0.25. However, when there are many experimental data, the weight value W may be determined by learning through learning module 230. In other words, if the user's stress level is already known from the response of the questionnaire, the weight value W may be determined through a simple linear regression method.

In a general stress level measuring system, since an EEG sensor, an ECG sensor, and an eye sensor have different measurement sensors, different characteristics may be extracted from each sensor or the characteristics may be learned and extracted using deep learning. At this time, in the general stress level measuring system, various characteristics may be extracted from raw data acquired from the sensor by any method and all of the characteristics are used.

On the contrary, there is a difference from the existing technology in that the system for analyzing the stress and managing the individual mental health according to an embodiment of the present invention may extract very various different characteristics in addition to information clearly known on the stress level and select a weight value capable of best confirming the stress level through learning. Accordingly, in the case of the present invention, since the characteristic dimension of the learned data is very large, the learning may be difficult, so that it is necessary to be a very sophisticated learning model (machine learning, deep learning model).

Here, according to the present invention, it is possible to design a model of predicting the stress and calculate a stress index based on the learned model after selecting a characteristic with the highest association with the stress level by learning using a recurrent neural network (RNN) or a long short term memory (LSTM).

Further, the diagnostic module 220 may also predict the stress by comparing a change in stress measurement information with standard stress information generated by the calibration module 140. Here, the stress measurement information means information including a stress index, concentration, sincerity, etc. measured from the user viewing the VR content and the stress guiding screen after performing the calibration step.

Method for Performing Stress Relaxation Content

Thereafter, when the stress analysis result is significantly higher than a predetermined stress level, a relaxation content according to the stress analysis result is performed (S540). More specifically, the output module 260 may output a content including various information according to the stress analysis result as a result screen (S630). For example, the relaxation content may include sounds, images, or videos as the content provided to lower the stress index of the user.

Also, the relaxation content may be output differently by the user or by the user's stress level.

The control module 250 may control the signal processing module 210, the diagnostic module 220, the learning module 230, and the output module 260.

Time Synchronization Method for a Series of Signals

Further, in order to analyze signals such as EEG, eyes, ECG, safety, EMG, etc. as various biosignals sensed by the HMD device 100, changes in EEG, eyes, ECG, safety, EMG, etc. of the user are measured for a very short time of at least 300 ms or less. In this case, a clock time of the HMD device 100 for displaying the stress guiding screen and a clock time of the biometric sensor that acquires biometric information of the user may be different from each other, or a clock time of the biometric sensor and a clock time of a processor of analyzing the biometric information may be different from each other.

As a result, the system for analyzing the stress and managing the individual mental health may perform time synchronizing for a series of signals by using at least two or more synchronization sensing signals so as to correctly analyze a change in biometric information according to user's video viewing.

Specifically, the mental care server 200 of the present invention receives a first synchronization sensing signal related to a first sensing signal (EEG sensing signal) received from a first biosignal sensor and receives a second synchronization sensing signal related to a second sensing signal (ECG sensing signal) received from a second biosignal sensor. Although described below, in the present invention, preferably, it will be understood that an event trigger signal is expressed based on the first synchronization sensing signal and the second synchronization sensing signal.

Here, the first synchronization sensing signal and the second synchronization sensing signal may be associated with at least two series of signals, respectively. For example, a series of signals may include at least one of an EEG sensing signal, an ECG sensing signal, a virtual reality image or a video signal, or various signals in the system.

Further, the first biosignal sensor and the second biosignal sensor may be at least one of a motion sensor that outputs a synchronization sensing signal representing motion information of the user, an illumination sensor that outputs a synchronization sensing signal representing ambient brightness information, an optical sensor that outputs a synchronization sensing signal representing light information of a predetermined light amount, and a sonic sensor that outputs a synchronization sensing signal representing predetermined audio information.

Further, the mental care server 200 receives a first synchronization sensing signal induced from the event trigger signal and received from the first biosignal sensor, receives a second synchronization sensing signal induced from the event trigger signal and received from the second biosignal sensor, calculates time difference information of the first synchronization sensing signal and the second synchronization sensing signal based on a time when the event trigger signal is expressed, and may synchronize the first biosignal sensor and the second biosignal sensor based on the time difference information. For example, the event trigger signal is a signal generated when the stimulation is given to the user, and preferably understood as a signal generated when the familiar/unfamiliar pictures to the user are randomly exposed or a short sound (beep) of a high-sound band is given as acoustic stimulation.

In other words, the event trigger signal may be displayed on a display of the HMD device 100 by randomly arranging the familiar pictures and the unfamiliar pictures, and may be a high-sound band (beep) having a range of about 10 to 90 db when a range of the general stimulation is at most about 500 to 10,000 hz, and may be a blinking screen displayed on the display of the HMD device 100.

Hereinafter, when the event trigger signal is detected, it will be described in two cases.

First, in the present invention, when the EEG sensing signal by the visual stimulation is detected, the time of the system may be calibrated so that a time expressing the event trigger signal (time when the stimulation is actually given) and a time when the ERP stimulation appears (time of the EEG sensor, that is, time when the event-related potential is measured) are the same as each other.

According to the present invention, after the event trigger signal is expressed, a time when the event trigger signal is expressed within a predetermined time and a time when the ERP stimulation (the first synchronization signal or the second synchronization signal) appears are measured, respectively. Thereafter, when the two measured signals are different from each other, the time may be calibrated so that a difference in the two times becomes the same. For example, since the event-related potentials (ERPs) when seeing the familiar picture by randomly exposing a familiar picture and an unfamiliar picture to the user are different from each other, time differences corresponding to the ERPs for the familiar picture and the unfamiliar picture are measured, respectively, to calibrate the time. However, the ERPs when seeing the familiar picture and the unfamiliar picture should have a difference of a predetermined level or more, but there may be no difference. Therefore, in this case, the accuracy of the measurement may be increased by re-combining the random arrangement of familiar pictures and unfamiliar pictures.

In addition, the present invention may calibrate the time based on a predicted ERP stimulation pattern by randomly exposing the familiar pictures and unfamiliar pictures to the user.

Generally, when the visual stimulation of a specific frequency region on the screen is exposed to the user, there is a phenomenon that the user's EEG is synchronized in accordance with the corresponding frequency. That is, the user's EEG may be synchronized in accordance with the corresponding frequency. Accordingly, in the case where it is assumed that the corresponding region is viewed by the user when any portion of the screen, for example, a blinking screen with 60 Hz is displayed (at a level that the user is not recognized), on the system of the present invention, whether the user views the region thereof may be confirmed. The time of the system may be calibrated so that the EEG synchronization time (EEG sensor time) and a time (mobile time) when the content is reproduced are the same as each other.

Further, according to the present invention, in the case of detecting the EEG sensing signal by audio stimulation, when a short sound (beep) of a high-sound band is given as auditory stimulation, the time synchronization may be performed by using that the EEG response to the corresponding stimulation is immediately shown. However, even if the time synchronization is accurately matched, in the case of synchronization between internal sensor systems, an error according to the time is rarely caused, but in the case of synchronization between mobile or third devices to which the content is reproduced, a delay error may also occur according to a network state. Accordingly, the present invention may confirm a time synchronization error by exposing a signal detection method optionally in the middle of the content, and perform the time calibration.

Therefore, a HDM device 900 according to another embodiment of the present invention performs time synchronization for a series of signals using two synchronization sensors to calibrate a time error between components in the system and a time error between different systems, thereby improving the accuracy of the measurement.

Although the exemplary embodiments of the present invention have been described in detail with reference to the accompanying drawings, the present invention is not limited thereto and may be embodied in many different forms without departing from the technical concept of the present invention. Therefore, the exemplary embodiments disclosed in the present invention are intended not to limit the technical spirit of the present invention but to describe the present invention and the scope of the technical spirit of the present invention is not limited by these exemplary embodiments. Therefore, it should be understood that the above-described exemplary embodiments are illustrative in all aspects and do not limit the present invention. The protective scope of the present invention should be construed based on the appended claims, and all the technical spirits in the equivalent scope thereof should be construed as falling within the scope of the present invention.

What is claimed is:

1. A method for analyzing stress and managing individual mental health using a HMD device, the method comprising:
a calibration step for generating standard stress information by calibrating biosignals received from a plurality of biosignal sensors;
a stress measurement content performance step for generating a stress guiding screen, measuring biometric data of a user via the generated stress guiding screen, and calculating stress measurement information of the user by comparing the measured biometric data with at least one of the standard stress information and the biosignals; and
a stress analysis content performance step for extracting a plurality of characteristics from the biometric data, and predicting a stress index of the user based on the plurality of extracted characteristics,
wherein the standard stress information includes an initial stress index, and a reference value for a specific emotion.

2. The method of claim 1, wherein in the stress analysis content performance step, the extracted characteristic is substituted with a stress level to measure the stress index of the user, and
the stress level is calculated by the following Equation 1.
Stress level=EG1*Weeg1 . . . + . . . EEGN*WeegN+EGG1*Wecg1 . . . + . . . ECGM*WecgM+EYE1*Weye1 . . . + . . . EYEK*WeyeK [Equation 1]
(here, W represents a weight value of each of an EEG sensor, an ECG sensor, and an eye sensor.)

3. The method of claim 1, wherein in the stress analysis content performance step, at least one of the stress index and the emotion of the user is analyzed by comparing a difference in the stress measurement information based on the standard stress information.

4. The method of claim 1, wherein in the stress analysis content performance step, the stress index is predicted based on the extracted characteristics using a recurrent neural network (RNN) or a long short term memory (LSTM).

5. The method of claim 1, further comprising:
a stress relaxation content performance step of generating a stress relaxation content according to the stress analysis result,
wherein the stress relaxation content is output in at least one form of sounds, images, and videos, and is provided with a different content depending on the user or user's stress index.

6. The method of claim 1, wherein the plurality of biosignal sensors include first and second biosignal sensors,
the method, further comprising:
receiving a first synchronization sensing signal induced from an event trigger signal and received from the first biosignal sensor;
receiving a second synchronization sensing signal induced from the event trigger signal and received from the second biosignal sensor; and
calculating time difference information of the first synchronization sensing signal and the second synchronization sensing signal based on a time when the event trigger signal is expressed, and synchronizing the first and second biosignal sensors based on the time difference information.

7. The method of claim 6, wherein the event trigger signal is displayed on a display of the HMD device by randomly arranging familiar pictures and unfamiliar pictures.

8. The method of claim 6, wherein the event trigger signal includes a beep sound.

9. The method of claim 6, wherein the event trigger signal is a blinking screen displayed on a display of the HMD device.

10. A system for analyzing stress and managing individual mental health using a HMD device, the system comprising:
a HMD device which measures biosignals from a plurality of biosignal sensors; and
a mental care server which receives the measured biosignals and calculates stress measurement information based on the received biosignals,
wherein the mental care server generates standard stress information by calibrating the biosignals, generates a stress guiding screen, measures biometric data of a user via the stress guiding screen, calculates the stress measurement information of the user by comparing the measured biometric data with at least one of the standard stress information and the biosignals, extracts characteristics from the biometric data, and predicts a stress index of the user based on the extracted characteristics,
wherein the standard stress information includes an initial stress index, and a reference value for a specific emotion.
